SEQUENCE LISTING (6 sequences)

SEQ ID NO: 1 - protein, 12 aa, synthetic construct
HTHGAARVPDHR

SEQ ID NO: 2 - protein, 7 aa, synthetic construct
LGSKPIN

SEQ ID NO: 3 - DNA, 90 bp, synthetic construct
  source 1..90: mol_type = other DNA
atatataagctttgcccatactcacggcgccgcccgcgttccggatcatcgcggtggaggtgcagaaggtgatgacccggctaaagctgc

SEQ ID NO: 4 - DNA, 33 bp, synthetic construct
  source 1..33: mol_type = other DNA
ggcaaagcttagcataggaacaagagtcgcaac

SEQ ID NO: 5 - DNA, 75 bp, synthetic construct
  source 1..75: mol_type = other DNA
atatataagctttgccctgggttctaaaccgattaacggtggaggtgcagaaggtgatgacccggctaaagctgc

SEQ ID NO: 6 - DNA, 33 bp, synthetic construct; identical to SEQ ID NO: 4
  source 1..33: mol_type = other DNA

(19) United States
(12) Patent Application Publication
(10) Pub. No.: US 2024/0353411 A1
(43) Pub. Date: Oct. 24, 2024

(54) HE4 DETECTING PEPTIDE AND USE THEREOF

(71) Applicant: Ajou University Industry-Academic Cooperation Foundation, Gyeonggi-do (KR)

(72) Inventors: Hyo Eon JIN, Gyeonggi-do (KR); Dongguk SHIN, Incheon (KR)

(73) Assignee: Ajou University Industry-Academic Cooperation Foundation, Gyeonggi-do (KR)

(21) Appl. No.: 18/687,744

(22) PCT Filed: Feb. 20, 2023
(86) PCT No.: PCT/KR2023/002385

§ 371 Date: Feb. 28, 2024

(30) Foreign Application Priority Data
Apr. 8, 2022 (KR) 10-2022-0044131
Nov. 11, 2022 (KR) 10-2022-0150153

(51) Int. Cl. G01N 33/574; C07K 7/06; C12N 7/00; C12Q 1/6886; G01N 21/78; G01N 33/544; G01N 33/553

(52) U.S. Cl. CPC G01N 33/57449; C07K 7/06; C12N 7/00; C12Q 1/6886; G01N 21/78; G01N 33/544; G01N 33/553

(57) ABSTRACT

The present invention relates to a peptide for detecting HE4 and use thereof and, more specifically, to a peptide for detecting HE4 that specifically binds to HE4 and use thereof for diagnosing ovarian cancer. The peptide that specifically binds to HE4, according to the present invention, can detect HE4 with high sensitivity and accuracy. In addition, when the peptide is expressed in the outer coat of a bacteriophage and constructed into a bacteriophage nano self-assembly, the peptide can conveniently detect HE4 from a sample through observation of a color change with a simple imaging device such as a smartphone, and in particular, the peptide can detect HE4 included in a small amount in saliva, and thus has the advantage of being very effectively used in early diagnosis of ovarian cancer.

### TECHNICAL FIELD

The present invention relates to a peptide for detecting HE4 and use thereof and, more specifically, to a peptide for detecting HE4 that specifically binds to HE4 and use thereof for diagnosing ovarian cancer.

### BACKGROUND

Due to rapid aging, the incidence of cancer is increasing worldwide and the number of deaths resulting from it is also rapidly increasing. Cancer is the number one cause of death, and the direct medical expenses spent by domestic cancer patients for cancer treatment amount to 2.3 trillion won (KRW) per year, and the resulting social and economic losses were reported to amount to 14.1 trillion won as of 2005. Accordingly, in order to mitigate economic losses caused by cancer, research is being intensively invested in early cancer diagnosis and treatment around the world.

From the WHO's medical perspective, it is reported that ⅓ of the population with cancer can be cured if diagnosed early, and the remaining ⅓ can also be alleviated. In addition, as a result of a cancer center survey at a hospital in Korea, it has been found that the 10-year survival rate for lung cancer when discovered in stage 1 increased significantly from 9% to 48.9%, and even stage 4 patients have a survival rate that doubles when diagnosed early, so the importance of early diagnosis of cancer disease is becoming more prominent over time.

However, existing cancer diagnosis methods mainly rely on endoscopy and biopsy, which are invasive and painful for patients, and screening methods that use expensive equipment for early diagnosis of disease require high medical costs, so new diagnostic methods with improved convenience and accessibility to patients are required.

Meanwhile, to solve these difficulties, various in vitro diagnostic devices for diagnosing prostate cancer, lung cancer, and breast cancer using blood, including HPV DNA chips for diagnosing cervical cancer, have been released, but cases where their effectiveness has been proven and commercialized are rare. In addition, although existing in vitro diagnostic kits are expensive products that require high test costs, they show low test reliability, making it difficult to accurately identify disease factors.

Among cancers, ovarian cancer is a malignant tumor that occurs in the ovaries and frequently occurs in menopausal women in their 50 s or older, and is the most common gynecological cancer in women, along with cervical cancer. According to data from the Health Insurance Review and Assessment Service in 2019, 47% of female patients who die from cancer die from ovarian cancer, showing a significantly higher fatality rate than other female cancers such as cervical cancer, breast cancer, and thyroid cancer. According to a report from the NIH (Cancer Stat Facts: Ovarian Cancer), more than 1.2% of women suffer from ovarian cancer, and the 5-year survival rate for ovarian cancer patients is only 49.1%.

In particular, ovarian cancer is often in the metastatic stage when symptoms appear, and according to actual reports, more than 70% of patients are diagnosed in the advanced stage (Holschneider and Berek, 2000), and about 60% are reported to be diagnosed after (distant) metastasis (Siegel et al., 2017; and NIH, SEER program). On the other hand, the 5-year survival rate upon diagnosis in the early stage (localized) increases to a very high level of 92.6%.

Therefore, the most effective strategy to enhance the survival rate and treatment possibilities for ovarian cancer is early diagnosis of ovarian cancer. Currently, clinical diagnosis involves confirming the tumor through physical examination methods using ultrasound or palpation, and then determine whether it is malignant through a biopsy or blood test, and so these methods are currently the earliest way to detect ovarian cancer. However, the method using ultrasound and palpation examination are highly invasive, causing significant pain and burden to patients because it can only confirm the tumor after it has grown to a certain extent, and a biopsy is essential for accurate diagnosis of malignant tumor. A blood test was designed as a diagnostic method to replace these methods, and through much investment and research to discover blood biomarkers for diagnosing ovarian cancer, biomarkers such as CA125 and HE4 were reported, but due to low sensitivity and specificity, each biomarker is used only for reference in clinical trials, and it is difficult to replace existing methods for diagnosing ovarian cancer.

The most common biomarker for early diagnosis of ovarian cancer is CA125. However, in addition to ovarian cancer, CA125 is also detected in ovarian adenomyosis, uterine myoma, endometrial pathology, and endometriosis, limiting its use as a single biomarker. Therefore, when additional biomarkers for ovarian cancer diagnosis, such as HE4, are used together, the accuracy of diagnosis can be increased. However, methods for detecting biomarkers other than CA125 still have very low sensitivity and specificity (Baron et al., 2003; Perkins et al., 2003).

The ROMA score, approved by the FDA in 2011, is a combination of HE4, CA125, and menopause and has a specificity of about 75%. Another biomarker-based index, called the Copenhagen Index (CPH-I), which uses a combination of HE4, CA125, and age, was developed by Karlsen et al. and is similar to ROMA but does not take into account ultrasound and menopausal status. The multiple biomarker-based analysis methods currently used in clinical practice described above have low specificity and sensitivity, and includes, in addition to plasma markers, confirmation of pelvic masses through ultrasound, menopause status, and age, etc. as markers, so they cannot be used as a diagnostic method for ovarian cancer for all women, and therefore, the above methods are used as a primary classification and reference test method for patients rather than an ideal diagnostic method. Therefore, in order to confirm the diagnosis of ovarian cancer, a conventional biopsy must also be performed, which still causes pain and burden on the patient.

As a result, there are currently no biomarkers capable of diagnosing ovarian cancer with high specificity and sensitivity, especially early diagnosis in stages I and II, despite the high need for them. In addition, the combination of biomarkers does not always involve improvement in sensitivity and specificity, and even if one of sensitivity and specificity improves, the other often decreases. Therefore, there is a need to develop a method that enables early diagnosis of ovarian cancer with high sensitivity and specificity, as well as a diagnostic method using the optimal combination of these methods.

Under this background technology, the present inventors have made diligent efforts to develop a technology that can diagnose ovarian cancer with high specificity and sensitivity, especially by a simple method using saliva, etc., and as a result, were able to discover a peptide that specifically binds to CA125, a known ovarian cancer biomarker, and a peptide that specifically binds to HE4, a known ovarian cancer biomarker, and have confirmed that when these are expressed in large quantities on bacteriophages, developed into nano self-assemblies, and used as biosensors, CA125 or HE4 contained in saliva can be detected in a very simple method with high accuracy and sensitivity, and completed the present invention.

### SUMMARY OF THE INVENTION

### Technical Problem

The present invention is directed to providing a peptide that specifically binds to HE4 and its use in diagnosing ovarian cancer.

### Technical Solution

In order to achieve the above objects, the present invention provides a peptide represented by SEQ ID NO: 2 that specifically binds to HE4.

In the present invention, it may be characterized in that the peptide is a peptide for detecting ovarian cancer.

The present invention also provides a composition for diagnosing ovarian cancer, comprising a peptide represented by SEQ ID NO: 2 that specifically binds to HE4.

The present invention also provides a bacteriophage in which the peptide represented by SEQ ID NO: 2 that specifically binds to HE4 is externally expressed.

In the present invention, it may be characterized in that the bacteriophage is M13 or F88 phage.

The present invention also provides a bacteriophage nano self-assembly produced by loading a substrate in the suspension of the bacteriophage and pulling the substrate vertically at a speed of 30 to 60 μm/min.

In the present invention, it may be characterized in that the substrate is a gold-coated silicon substrate.

The present invention also provides a method for providing information for diagnosing ovarian cancer, comprising the following steps:
(a) contacting a sample separated from a subject with a nano self-assembly of a bacteriophage in which a peptide represented by SEQ ID NO: 2 is externally expressed;
(b) analyzing a color change pattern of the bacteriophage nano self-assembly of step (a); and
(c) comparing the color change pattern of step (b) with the color change pattern of a control group to determine whether ovarian cancer has occurred.

In the present invention, it may be characterized in that the sample is selected from the group consisting of whole blood, leukocytes, peripheral blood mononuclear cells, buffy coat, plasma, serum, sputum, tears, mucus, nasal washes, nasal aspirate, breath, urine, semen, saliva, peritoneal washings, ascites, cystic fluid, meningeal fluid, amniotic fluid, glandular fluid, pancreatic fluid, lymph fluid, pleural fluid, nipple aspirate, bronchial aspirate, synovial fluid, joint aspirate, organ secretions, cells, cell extract, and cerebrospinal fluid obtained from a separated organ, tissue, cell, or subject.

In the present invention, it may be characterized in that the color change pattern in step (b) is an RGB (Red, Green, Blue) pattern.

In the present invention, it may be characterized in that the RGB (Red, Green, Blue) pattern in step (b) is analyzed for the color change pattern after conversion to black and white.

### Advantageous Effects

The peptide that specifically binds to HE4, according to the present invention, can detect HE4 with high sensitivity and accuracy. In addition, when the peptide is expressed in the outer coat of a bacteriophage and constructed into a bacteriophage nano self-assembly, the peptide can conveniently detect HE4 from a sample through observation of a color change with a simple imaging device such as a smartphone, and in particular, the peptide can detect HE4 included in a small amount in saliva, and thus has the advantage of being very effectively used in early diagnosis of ovarian cancer.

### BRIEF DESCRIPTION OF THE DRAWINGS

FIG. 1 shows the results confirming that the two types of peptides newly discovered in the present invention specifically bind to CA125 and HE4, respectively.

FIG. 2A shows, using SDS-PAGE, the results of confirming that the peptides of the present invention are expressed in F88 bacteriophages (F88-CA125, F88-HE4) constructed to express each of the two types of peptides newly discovered in the present invention.

FIG. 2B shows, using MALDI-TOF, the results of confirming that the peptides of the present invention are expressed in F88 recombinant bacteriophages (F88-CA125, F88-HE4) constructed to express each of the two types of peptides newly discovered in the present invention.

FIG. 3 shows a sensing platform produced with F88-CA125 phage.

FIG. 4 shows a sensing platform produced with F88-HE4 phage.

FIG. 5 shows the results of analyzing RGB patterns using a smartphone application for detecting CA125 in saliva using a sensing platform produced with F88-CA125 phage.

FIG. 6 shows the results of analyzing RGB patterns using a smartphone application for detecting HE4 in saliva using a sensing platform produced with F88-HE4 phage.

FIG. 7 shows a sensing platform produced with a mixture of F88-CA125 phage and F88-HE4 phage.

FIG. 8 shows the results of analyzing RGB patterns using a smartphone application for detecting HE4 in saliva using a sensing platform produced with a mixture of F88-CA125 phage and F88-HE4 phage.

### DETAILED DESCRIPTION OF THE EMBODIMENTS

Unless otherwise defined, all technical and scientific terms used in this specification have the same meaning as commonly understood by a person skilled in the art to which the present invention pertains. In general, the nomenclature used in this specification is well known and commonly used in the art.

In the concentration range described in this specification, “to” is used to mean including both critical ranges (more than or equal to and less than or equal to), and when both critical ranges are not included, the concentration range is described as “more than” and “less than.” In this specification, “about” used in numerical values is used to include a range expected to produce substantially the same effect as the numerical value described by a person skilled in the art, and for example, may mean ±20%, ±10%, ±5%, etc. of the stated numerical value, but is not limited thereto.

While ovarian cancer affects approximately 1 to 2% of women, 47% of female patients who die from cancer are reported to have ovarian cancer, showing a significantly higher mortality rate than other female cancers. In addition, the 5-year survival rate for ovarian cancer patients diagnosed with ovarian cancer is very low, less than 50%. The reason for such a high mortality rate of ovarian cancer is that ovarian cancer is often diagnosed when the cancer is already at an advanced stage, and most symptoms appear after the metastatic stage.

Therefore, the most effective strategy to enhance the survival rate and treatment possibilities of ovarian cancer is early diagnosis of ovarian cancer. However, the current clinical approach involves confirming the tumor through physical examination methods using ultrasound or palpation, and then determine whether it is malignant through a biopsy or blood test. While diagnosiing ovarian cancer through a blood test is non-invasive and reduces the burden on patients compared to biopsy, etc., but has the problem of requiring specialized equipment or taking a long time for the test when trying to confirm the expression level of CA125 and HE4 in the blood using a PCR test, and when trying to confirm with an antigen test, effective antibodies that can accurately confirm the expression level have not been developed, so a simple, rapid, and highly accurate early diagnosis method is needed.

Accordingly, in the present invention, novel peptide sequences that bind to cancer biomarkers CA125 and HE4, respectively, were discovered through phage display screening.

The term “Carbohydrate Antigen 125 (hereinafter referred to as CA125)” of the present invention is a high molecular weight glycoprotein, and is also referred to as MUC16 (mucin 16, cell surface associated). CA125 is a tumor antigen widely used in ovarian cancer monitoring, and using a rabbit polyclonal antibody produced against the purified CA125 antigen to screen cells from an ovarian cancer cell (OVCAR-3) cDNA library of E. coli, Yin and Lloyd (2001) cloned a long partial cDNA and designated it as MUC16. The deduced 1,890 amino acid proteins have an N-terminal region of nine partially conserved tandem repeats, a possible transmembrane region, and a potential tyrosine phosphorylation site. MUC16 is also high in leucine content. Northern blot analysis showed that the level of MUC16 mRNA correlated with the expression of CA125 in a panel of cell lines. In the present invention, the representative human CA125 sequence is the same as NCBI Accession No. NP_078966.2, but not limited thereto, and includes a protein or a mutation thereof, containing an amino acid sequence determined to be substantially homologous to the above sequence. For example, it may include, but is not limited to, a sequence having about 80% or more homology, preferably 85% or more homology, more preferably 90% or more homology, more preferably 95% or more homology, more preferably 97% or more homology, and most preferably 99% or more homology to the above sequence.

The term “HUMAN EPIDIDYMIS PROTEIN 4 (hereinafter referred to as HE4)” in the present invention is a human epididymal protein, and is also referred to as WFDC2 (WAP 4-DISULFIDE CORE DOMAIN 2). By rescreening an epididymal cDNA library with differential screening for human epididymis-specific transcriptomes, Kirchhoff et al. (1991) cloned WFDC2, called HE4. The deduced 125 amino acid proteins have an N-terminal signal sequence, two similar cysteine-rich domains, and a predicted N-glycosylation site. The predicted mature protein has 95 amino acids and a calculated molecular mass of 10 kDa. In the present invention, the representative human HE4 sequence is the same as NCBI Accession No. NP_006094.3, but not limited thereto, and includes a protein or a mutation thereof, containing an amino acid sequence determined to be substantially homologous to the above sequence. For example, it may include, but is not limited to, a sequence having about 80% or more homology, preferably 85% or more homology, more preferably 90% or more homology, more preferably 95% or more homology, more preferably 97% or more homology, and most preferably 99% or more homology to the above sequence.

Therefore, in one aspect, the present invention relates to a peptide represented by SEQ ID NO: 1 that specifically binds to CA125.

In another aspect, the present invention relates to a peptide represented by SEQ ID NO: 2 that specifically binds to HE4.

In the present invention, it may be characterized in that the peptide represented by SEQ ID NO: 1 and the peptide represented by SEQ ID NO: 2 are each a peptide for detecting ovarian cancer, but it is not limited thereto.

That is, the peptide represented by SEQ ID NO: 1 and the peptide represented by SEQ ID NO: 2 according to the present invention will be able to be used to detect various diseases using CA125 and HE4 as biomarkers, respectively.

Specifically, the peptide represented by SEQ ID NO: 1 can be used to diagnose gynecological cancers such as ovarian cancer as well as endometrial cancer. In addition, the peptide represented by SEQ ID NO: 1 can be used to diagnose pancreatic cancer, lung cancer, breast cancer, colon cancer, and gastrointestinal cancer. However, the use of the peptide represented by SEQ ID NO: 1 is not limited thereto, and it is obvious to those skilled in the art that it can be used for any purpose requiring detection of CA125.

For example, the peptide represented by SEQ ID NO: 1 can be used to determine the prognosis of endometrial cancer, determine the size and stage, determine prognosis, detect recurrence, monitor therapeutic effect, and predict survival rate of ovarian cancer.

In the present invention, it may be characterized in that if CA125 is detected at 3000 U/mL or more in saliva, it is judged that the patient has developed ovarian cancer or is a risk group that is likely to develop ovarian cancer, but it is not limited thereto (see Chen DX, Schwartz PE, Li FQ. Saliva and serum CA 125 assays for detecting malignant ovarian tumors. Obstet Gynecol. 1990 April;75(4):701-4. PMID: 2179784).

In addition, the peptide represented by SEQ ID NO: 2 can be used to diagnose ovarian cancer, such as epithelial ovarian cancer, using CA125 detection together or alone. However, the use of the peptide represented by SEQ ID NO: 2 is not limited thereto, and it is obvious to those skilled in the art that it can be used for any purpose requiring detection of HE4.

For example, the peptide represented by SEQ ID NO: 2 can be used to determine the size and stage, determine prognosis, detect recurrence, monitor therapeutic effect, and predict survival rate of ovarian cancer.

In the present invention, it may be characterized in that if HE4 is detected at 150 pmole/L or more in saliva, it is judged that the patient has developed ovarian cancer or is a risk group that is likely to develop ovarian cancer, but it is not limited thereto.

Meanwhile, in yet another aspect, the present invention relates to a composition for diagnosing ovarian cancer comprising the peptide represented by SEQ ID NO: 1.

The present invention can be provided for the use of the peptide represented by SEQ ID NO: 1 in diagnosing ovarian cancer.

The present invention can also be provided for the use of the peptide represented by SEQ ID NO: 1 in the manufacture of an ovarian cancer diagnostic reagent.

The present invention can also be provided for a method for diagnosing ovarian cancer, comprising comparing the expression level of CA125 in a subject requiring ovarian cancer diagnosis with a control group using the peptide represented by SEQ ID NO: 1 to determine whether ovarian cancer has occurred.

In yet another aspect, the present invention relates to a composition for diagnosing ovarian cancer comprising the peptide represented by SEQ ID NO: 2.

The present invention can be provided for the use of the peptide represented by SEQ ID NO: 2 in diagnosing ovarian cancer.

The present invention can also be provided for the use of the peptide represented by SEQ ID NO: 2 in the manufacture of an ovarian cancer diagnostic reagent.

The present invention can also be provided for a method for diagnosing ovarian cancer, comprising comparing the expression level of HE4 in a subject requiring ovarian cancer diagnosis with a control group using the peptide represented by SEQ ID NO: 2 to determine whether ovarian cancer has occurred.

In yet another aspect, the present invention relates to a composition for diagnosing ovarian cancer comprising the peptide represented by SEQ ID NO: 1 and the peptide represented by SEQ ID NO: 2.

The present invention can be provided for the use of the composition in diagnosing ovarian cancer.

The present invention can also be provided for the use of the composition as an ovarian cancer diagnostic reagent.

The present invention can also be provided for a method for diagnosing ovarian cancer, comprising comparing the expression levels of CA125 and HE4 in a subject requiring ovarian cancer diagnosis with a control group using the composition to determine whether ovarian cancer has occurred.

In the present invention, “diagnosis” means accurately identifying the condition of a subject for a particular disease or disorder. For example, the condition of a subject for a particular disease or disorder is used in a broad sense including not only susceptibility to the particular disease or disorder, the determination of the disorder currently suffering from the subject, but also the identification of the characteristics of the disorder such as prognosis of the subject, the identification of the cancer condition, the determination of the stage of the cancer or the prediction of the susceptibility and responsiveness of the cancer to treatment; the acquisition of a basis for appropriate treatment according to the patient's disease and condition, such as confirming the subject's condition to identify the therapeutic efficacy of a specific drug; and furthermore, the prediction and identification of the recurrence in the subject cured of a particular disease or disorder. In the present invention, preferably, the diagnosis is to identify whether a disease has occurred or the possibility of developing the disease.

In the present invention, it may be characterized in that the disease or disorder to be diagnosed may be ovarian cancer. The ovarian cancer refers to a malignant tumor, that is, cancer that occurs in the ovaries or organs surrounding the ovaries. As specific examples, the ovarian cancer includes epithelial cell carcinoma, germ cell tumor, and sex cord-stromal tumor in the ovary, and as more specific examples, includes serous carcinoma, mucinous carcinoma, endometrioid carcinoma, clear cell carcinoma, malignant brenner tumor, undifferentiated cell carcinoma, and unclassified carcinoma in the ovary, but is not limited thereto.

In the present invention, the ovarian cancer can be classified according to stage, and specifically, it can be classified into stages 1 to 4 ovarian cancer, and stages 1 and 2 are classified as early stages, and stages 3 and 4 are classified as advanced stages (FIGO classification standards).

The term “stage of ovarian cancer” of the present invention can be classified according to the following criteria (TNM and FIGO classification criteria, 2019, Urogenital Imaging Diagnostic Gynecological Imaging, The Korean Society of Urogenital Radiology).

TABLE 1

| | | |
|---|---|---|
| Classification | TNM | FIGO |

Primary cancer (T)

| | | | |
|---|---|---|---|
| | TX | | Primary cancer not evaluated |
| | T0 | | No evidence of primary cancer |
| Stage 1 | T1 | I | Tumor confined to the ovaries (one or both) or fallopian tubes |
| | T1a | IA | Tumor confined to one ovary (intact capsule) or to the surface of |
| | | | the fallopian tubes; No malignant cells in ascites or peritoneal |
| | | | lavage fluid |
| | T1b | IB | Tumor confined to one or both ovaries (intact capsule) or to the |
| | | | surface of the fallopian tubes; No tumors on the surface of the |
| | | | ovaries or fallopian tubes; No malignant cells in ascites or |
| | | | peritoneal lavage fluid |
| | T1c | IC | Tumor confined to one or both ovaries or fallopian tubes and |
| | | | accompanied by some of the following: |
| | T1c1 | IC1 | Spill during surgery |
| | T1c2 | IC2 | Rupture of the capsule during surgery or tumor on the |
| | | | ovarian/fallopian tubal surface |
| | T1c3 | IC3 | Malignant cells in ascites or peritoneal lavage fluid |
| Stage 2 | T2 | II | Tumor in one or both ovaries and invaded the pelvis below the |
| | | | pelvic brim, or primary peritoneal cancer |
| | T2a | IIA | When tumor invades the uterus, fallopian tubes, or ovaries either |
| | | | directly or through peritoneal implantation |
| | T2b | IIB | When tumor invades other pelvic tissues either directly or through |
| | | | peritoneal implantation |
| Stage 3 | T3 | III | When tumor or primary peritoneal cancer confined to one or both |
| | | | ovaries or fallopian tubes accompanied by microscopically |
| | | | confirmed extrapelvic peritoneal metastasis or metastasis to the |
| | | | retroperitoneal cavity (pelvic or para-aortic) lymph nodes |
| | T3a | IIIA2 | May or may not have microscopic extrapelvic peritoneal |
| | | | metastasis and retroperitoneal cavity lymph node metastasis |
| | T3b | IIIB | May or may not have macroscopic peritoneal metastasis less than |
| | | | 2 cm in length and retroperitoneal cavity lymph node metastasis |
| | T3c | IIIC | May or may not have macroscopic peritoneal metastasis more |
| | | | than 2 cm in length and retroperitoneal cavity lymph node |
| | | | metastasis (including tumors that invade the surface without |
| | | | invading the liver or spleen parenchyma) |

Local lymph node (N)

| | | | |
|---|---|---|---|
| | NX | | Local lymph node not evaluated |
| | ND | | No local lymph node metastasis |
| | No(i+) | | Isolated tumor cells smaller than 0.2 mm found in local lymph |
| | | | nodes |
| Stage 3 | N1 | IIIA1 | Histologically confirmed retroperitoneal cavity lymph node |
| | | | metastasis |
| | N1a | IIIA1i | Less than 10 mm in length |
| | N1b | IIIA1ii | More than 10 mm in length |

Distant metastasis (M)

| | | | |
|---|---|---|---|
| Stage 4 | M0 | | No distant metastasis |
| | M1 | IV | There is distant metastasis |
| | M1a | IVA | Pleural effusion fluid with positive cytology |
| | M1b | IVB | Metastasis to liver/spleen parenchyma, metastasis to extra- |
| | | | abdominal organs (including inguinal and extra-abdominal lymph |
| | | | node metastases), metastasis that invades full wall thickness |

It may be characterized in that the composition for diagnosing ovarian cancer of the present invention is used to diagnose all ovarian cancers in stages 1 to 4, and preferably used to diagnose early stage 1 to 2 ovarian cancer, but it is not limited thereto.

Meanwhile, the present invention can be provided as a kit for diagnosing ovarian cancer.

Therefore, in yet another aspect, the present invention relates to a kit for diagnosing ovarian cancer comprising the peptide represented by SEQ ID NO: 1.

In yet another aspect, the present invention relates to a kit for diagnosing ovarian cancer comprising the peptide represented by SEQ ID NO: 2.

In yet another aspect, the present invention relates to a kit for diagnosing ovarian cancer comprising the peptide represented by SEQ ID NO: 1 and/or the peptide represented by SEQ ID NO: 2.

In terms of the kit of the present invention, definitions and embodiments of terms not described may share the same features as those described in terms of the composition for diagnosing ovarian cancer, unless otherwise stated.

In the present invention, it may be characterized in that the kit for diagnosing ovarian cancer comprises the composition for diagnosing ovarian cancer of the present invention.

In the present invention, the kit for diagnosing ovarian cancer may include one or more different component compositions, solutions, or devices suitable for the analysis method.

In the present invention, the kit may be a protein chip kit, a rapid kit, or a selected reaction monitoring/multiple reaction monitoring (SRM/MRM) kit.

In addition, the kit according to the present invention may be a diagnostic kit containing a peptide represented by SEQ ID NO: 1 as an agent for measuring CA125 protein level and/or a peptide represented by SEQ ID NO: 2 as an agent for measuring HE4 protein level. In this case, the agent for measuring the protein level can be made, for example, as a kit for detecting diagnostic markers containing the essential elements necessary to perform ELISA, and may include chromophores, enzymes (e.g., conjugated with peptides), and their substrates and the like. In addition, it may include antibodies specific for the quantitative control group protein.

Furthermore, the kit can quantitatively measure through the signal size of the detection label. This detection label may be selected from the group consisting of enzymes, fluorescent substances, ligands, luminescent substances, microparticles, redox molecules and radioisotopes, but is not necessarily limited thereto.

The selected reaction monitoring (SRM) is also called multiple reaction monitoring (MRM), is a method used in tandem mass spectrometry and is used for targeted quantitative proteomic analysis. SRM used for targeted quantitative proteomic analysis is described in detail in Nature Methods. 9 (6): 555-566.

Meanwhile, in the present invention, the discovered novel peptide sequence is introduced into M13 bacteriophage using genetic engineering technology, and furthermore, in order to improve the binding ability to the biomarker and strengthen the detection intensity, the peptide represented by SEQ ID NO: 1 and the peptide represented by SEQ ID NO: 2 were each introduced into the gene of F88 bacteriophage to produce two types of functional bacteriophages in which the peptides were expressed on the outer coat of a bacteriophage.

Therefore, in yet another aspect, the present invention relates to a bacteriophage in which the peptide represented by SEQ ID NO: 1 is expressed on the outer coat, and in yet another aspect, relates to a bacteriophage in which the peptide represented by SEQ ID NO: 2 is expressed on the outer coat.

In the present invention, it will be apparent to those skilled in the art that the bacteriophage may be produced as a bacteriophage in which both the peptide represented by SEQ ID NO: 1 and the peptide represented by SEQ ID NO: 2 are expressed externally.

In the present invention, it may be characterized in that the bacteriophage is M13, T4, T7, λ, fd, fUSE, or F88 phage, but it is not limited thereto.

Meanwhile, in the present invention, a self-assembled nanostructure of F88 bacteriophage was produced by adjusting chemical and physical variables such as pH and electrolyte of the solvent of the solution in which the functional bacteriophage was dispersed, and a detection system showing high selectivity for ovarian cancer biomarkers CA125 and/or HE4 was implemented.

Therefore, in yet another aspect, the present invention relates to a phage nano self-assembly produced by loading a substrate in a suspension of a bacteriophage in which the peptide represented by SEQ ID NO: 1 is expressed on the outer coat, and pulling the substrate vertically at a speed of 30 to 60 μm/min.

In yet another aspect, the present invention relates to a phage nano self-assembly produced by loading a substrate in a suspension of a bacteriophage in which the peptide represented by SEQ ID NO: 2 is expressed on the outer coat, and pulling the substrate vertically at a speed of 30 to 60 μm/min.

As an aspect, the phage nano self-assembly may use a syringe pump to load a gold-coated Si wafer on an eppendorf tube containing a bacteriophage suspension, and then gradually increase the speed of the support on which the wafer is fixed to 30 to 60 μm/min, and coat the phage on the wafer while taking it out.

In the present invention, the suspension is prepared by suspending the bacteriophage in a buffer solution, and it may be characterized in that the buffer solution is a TBS (tris buffered saline) solution, but it is not limited thereto.

In the present invention, it may be characterized in that the substrate is a gold-coated silicon substrate, but it is not limited thereto.

In the present invention, the suspension may be prepared by diluting in 50 mM TBS buffer (50 mM Tris-HCl, 150 mM NaCl, pH 7.5) containing 0.01-0.2% Tween to achieve a phage concentration of 4-8 mg/mL, and preferably, may be prepared as a solution in TBS buffer (12.5 mM Tris-HCl, 37.5 mM NaCl, pH 7.5) containing 0.05% Tween to achieve a phage concentration of 6 mg/mL.

Meanwhile, in yet another aspect, the present invention relates to a method for providing information for diagnosing ovarian cancer, comprising the following steps:
(a) contacting a sample separated from a subject with a nano self-assembly of a bacteriophage in which a peptide represented by SEQ ID NO: 1 is expressed on its outer coat;
(b) analyzing a color change pattern of the bacteriophage nano self-assembly of step (a); and
(c) comparing the color change pattern of step (b) with the color change pattern of a control group to determine whether ovarian cancer has occurred.

In yet another aspect, the present invention relates to a method for providing information for diagnosing ovarian cancer, comprising the following steps:
(a′) contacting a sample separated from a subject with a nano self-assembly of a bacteriophage in which a peptide represented by SEQ ID NO: 2 is expressed on its outer coat;
(b) analyzing a color change pattern of the phage nano self-assembly of step (a′); and
(c) comparing the color change pattern of step (b) with the color change pattern of a control group to determine whether ovarian cancer has occurred.

In yet another aspect, the present invention relates to a method for providing information for diagnosing ovarian cancer, comprising the following steps:
(a) contacting a sample separated from a subject with a nano self-assembly of a bacteriophage in which a peptide represented by SEQ ID NO: 1 is expressed on its outer coat;
(a′) contacting a sample separated from a subject with a nano self-assembly of a bacteriophage in which a peptide represented by SEQ ID NO: 2 is expressed on its outer coat;
(b) analyzing a color change pattern of the phage nano self-assembly of step (a);
(b) analyzing a color change pattern of the phage nano self-assembly of step (a′); and
(c) comparing the color change patterns of steps (b) and (b′) with a color change pattern of a control group, respectively, to determine whether ovarian cancer has occurred.

In the above method, steps (a) to (b′) may be performed in any order. For example, step (a′) or step (a′) and step (b′) may proceed before step (a), and step (b) may proceed before step (a′).

In the present invention, ovarian cancer can be diagnosed by detecting CA125 using the peptide represented by SEQ ID NO: 1 or by detecting HE4 using the peptide represented by SEQ ID NO: 2, respectively, but if CA125 and HE4 are simultaneously detected using the peptide represented by SEQ ID NO: 1 and the peptide represented by SEQ ID NO: 2, there is an advantage in improving the accuracy of diagnosing ovarian cancer.

In the present invention, the sample may be whole blood, leukocytes, peripheral blood mononuclear cells, buffy coat, plasma, serum, sputum, tears, mucus, nasal washes, nasal aspirate, breath, urine, semen, saliva, peritoneal washings, ascites, cystic fluid, meningeal fluid, amniotic fluid, glandular fluid, pancreatic fluid, lymph fluid, pleural fluid, nipple aspirate, bronchial aspirate, synovial fluid, joint aspirate, organ secretions, cells, cell extract, or cerebrospinal fluid obtained from an isolated organ, tissue, cell, or subject, but is not limited thereto.

In the present invention, the sample may preferably be saliva.

In the present invention, it may be characterized in that the color change pattern in step (b) is an RGB (Red, Green, Blue) pattern, but it is not limited thereto.

In the present invention, it may be characterized in that the RGB (Red, Green, Blue) pattern in step (b) is analyzed for the color change pattern after conversion to black and white, but it is not limited thereto.

In the present invention, it may be characterized in that the step of determining whether ovarian cancer has occurred by comparing the color change pattern of the subject's sample with the color change pattern of the control group according to the present invention is interpreted by a prediction or classification model. In the present invention, it may be characterized in that the prediction or classification model is learned using a known data analysis method. For example, the prediction or classification model may be one learned using methods such as linear regression, logistic regression, ridge regression, lasso regression, jackknife regression, decision tree, random forest, K-means clustering, cross-validation, artificial neural network, ensemble learning, naive bayesian classifier, collaborative filtering, principal component analysis (PCA), and support vector machine (SVM), and may preferably be one learned using random forest or support vector machine, but is not limited thereto. In the present invention, in addition to known models, the prediction or classification model may be one learned by a supervised or unsupervised algorithm newly designed by a person skilled in the art for diagnosis of ovarian cancer based on embodiments of the present invention.

### EXAMPLES

Hereinafter, the present invention will be described in more detail through examples. These examples are only for illustrating the present invention, and it will be obvious to those skilled in the art that the scope of the present invention is not to be construed as limited by these examples.

### Example 1. Discovery of Peptide Sequences that Specifically Bind to CA125 or HE4

Phage display screening was repeated 3 to 4 times to discover peptide sequences that bind to CA125 or HE4.

### 1-1. Biopanning Round 1

CA125 (R&D systems) protein and HE4 (abcam) protein, which are salivary biomarkers for ovarian cancer, were purchased and used. Each biomarker was diluted in coating buffer (0.1 M NaHCO₃) and the proteins were coated on a 96-well plate. After treating the wells coated with CA125 or HE4 protein with blocking buffer (0.1 M NaHCO₃, 5 mg/mL BSA) to inhibit non-specific binding of proteins other than CA125 or HE4 protein, the peptide library (10¹¹ phages) (New England Biolabs, MA, USA) was processed, and incubated at room temperature. After removing unbound and weakly bound phages with a washing buffer (PBS-T buffer, 0.1% Tween 20), strongly bound phages were extracted with an extraction buffer (0.2 M Glycine-HCl, 1 mg/mL BSA). The phage extract was placed in a new eppendorf tube and neutralized by adding 1 M Tris-HCl. This was diluted 10 to 100 times, mixed with XL1-Blue bacteria (Agilent, CA, USA) and top agar (25 g LB medium, 7 g Agarose, 1 g MgCl₂, H₂O, Thermo Fisher Scientific, MA, USA), and was dispensed onto an LB/IPTG/X-gal plate, and the number of phages in the phage extract was confirmed.

### 1-2. Phage Amplification and Purification

After the phage extract was amplified by culturing with bacteria in LB medium, centrifuged (10000 rpm, 10 minutes), and 80% of the supernatant was transferred to a new sterilized eppendorf tube, 20% PEG/2.5 M NaCl was added to ⅙ the volume of the supernatant and incubated at 4° C. to precipitate phages. The centrifugation process was repeated again to obtain purified phage, and the phage concentration was measured by UV and used in the next round.

### 1-3. Biopanning Rounds 2 to 4

The process was the same as Round 1, and the next round was conducted by gradually increasing the concentration of Tween20 in PBS-T to a higher concentration (0.2, 0.4, 0.5% Tween20) than the 0.1% in round 1 during the washing process. Through this process, phages displaying peptide sequences with high binding affinity to CA125 or HE4 proteins were obtained.

### 1-4. Sequence Analysis of Peptides through DNA Sequencing

By analyzing the DNA sequence of M13 phage obtained as a screening result, a peptide sequence (HTHGAARVPDHR, SEQ ID NO. 1) that binds to CA125 protein and a peptide sequence (LGSKPIN, SEQ ID NO. 2) that binds to HE4 protein were discovered.

### 1-5. Evaluation of HE4 Binding Affinity and Specificity of Discovered Peptides

In order to evaluate the binding affinity and specificity of the discovered SEQ ID NO: 1 peptide to the CA125 protein, and the binding affinity and specificity of the SEQ ID NO: 2 peptide to HE4, ELISA assay was performed according to the manufacturer's instructions using CA125 protein (R&D Systems Inc, MN, USA), HE4 protein (Sinobiological, PA, USA), HRP-conjugated M13 antibody (Sinobiological, PA, USA), and TMB solution (Thermo Scientific, MA, USA). In addition, to measure the binding affinity K_{d} (dissociation constant), the discovered peptides of SEQ ID NO: 1 and SEQ ID NO: 2 were synthesized (Biostem, Suwon, Korea), CA125 protein and HE4 protein were each fluorescently labeled using Protein Labeling kit RED-NHS 2nd Generation (NanoTemper Technologies, Munich, Germany). Afterwards, the binding affinity between peptide and protein was measured using Monolith NT.115 (NanoTemper Technologies, Munich, Germany) according to the manufacturer's instructions.

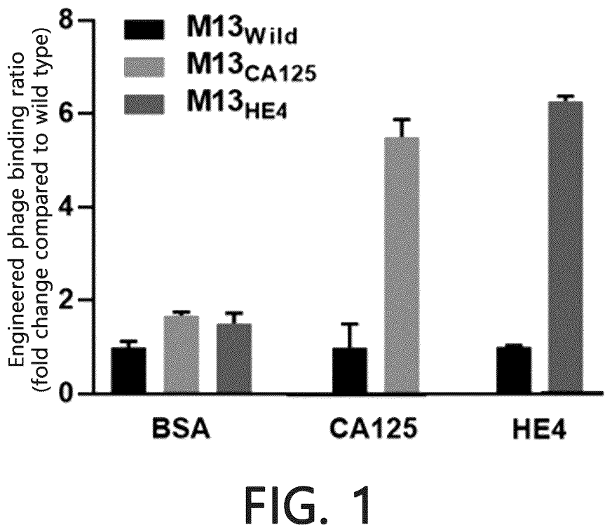

As a result, it was confirmed that the peptide with SEQ ID NO: 1 specifically bound to the CA125 protein, and the peptide with SEQ ID NO: 2 specifically bound to the HE4 protein (FIG. 1). Specifically, the peptide with SEQ ID NO: 1 was confirmed to bind to the CA125 protein with a binding affinity of K_{d}=11.44±4.23 nM, and the peptide with SEQ ID NO: 2 was confirmed to bind to the HE4 protein with a binding affinity of K_{d}=15.34±12.00 μM.

### Example 2. Development of Functional Phages

Since n M13 phage, 5 copies of the discovered peptide are expressed in the minor coat, but in order to be used as a sensor bioreceptor, a larger number of peptides must be expressed to be advantageous in terms of sensitivity, through genetic recombination of F88 phage, the peptide of SEQ ID NO: 1 or peptide of SEQ ID NO: 2 was introduced into the major coat of F88 phage to produce recombinant F88 phages (F88-CA125 phage and F88-HE4 phage, respectively).

Specifically, using the F88 vector as a template and the Phusion High-Fidelity DNA Polymerase set (New England Biolabs, MA USA) as the following primer pair, PCR amplification was performed under the following conditions: 95° C. for 1 minute, (95° C. for 30 seconds, 59° C. for 30 seconds, 7° C. for 3 minutes)×30 cycles, and 72° C. for 10 minutes.

F88-CA125:
5′-ATATATAAGCTTTGCCCATACTCACGGCGCCGCCCGCGTTCCGGATC
ATCGCGGTGGAGGTGCAGAAGGTGATGACCCGGCTAAAGCTGC-3′,
5′-GGCAAAGCTTAGCATAGGAACAAGAGTCGCAAC-3′,
F88-HE4:
5′-ATATATAAGCTTTGCCCTGGGTTCTAAACCGATTAACGGTGGAGGTG
CAGAAGGTGATGACCCGGCTAAAGCTGC-3′, 5′-GGCAAAGCTTAGCA
TAGGAACAAGAGTCGCAAC-3′

After inserting each gene into the F88 phage vector (Smith group, University of Missouri, MO, USA), the PCR products were purified by agarose gel electrophoresis. The purified product was treated with Hind III restriction enzyme, ligated using T4 DNA ligase at 16° C. for 18 hours, and then transformed into XL1-Blue bacteria to obtain a phage clone.

The two recombinant F88 phages were cultured in NZY medium supplemented with 1 mM IPTG to express each peptide, and the amplified phage was precipitated by adding polyethylene glycol (PEG) solution (20% (w/v) PEG8000, 2.5M NaCl) and resuspended by adding buffer to the phage precipitate, and then used.

Afterwards, SDS-PAGE and MALDI-TOF analysis were performed.

For SDS-PAGE, phage mixed with sample buffer and denatured at 90° C. for 10 minutes was loaded on a 16% polyacrylamide gel and electrophoresed using the standard Tricine-SDS-PAGE buffer. Afterwards, the gel was stained with Imperial™ protein stain (Thermo scientific, MA, USA) for 1 hour, and the stained protein was confirmed by destaining with DI water.

It was attempted to confirm peptide expression in recombinant pVIII using MALDI-TOF mass spectrometry, so 2 μL of 1 mg/mL phage suspended in DDW was mixed with 2 μL of matrix solution (10 mg/mL of Sinapinic acid (SA) in 0.1% TFA/ACN (1:1, v/v)), dropped on the substrate, dried in vacuum, and analyzed with UltraflexIIITOF/TOF (Bruker Daltonics, MA, USA).

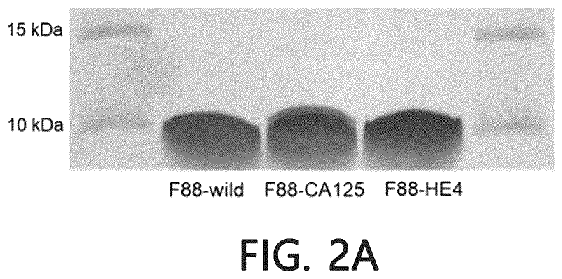

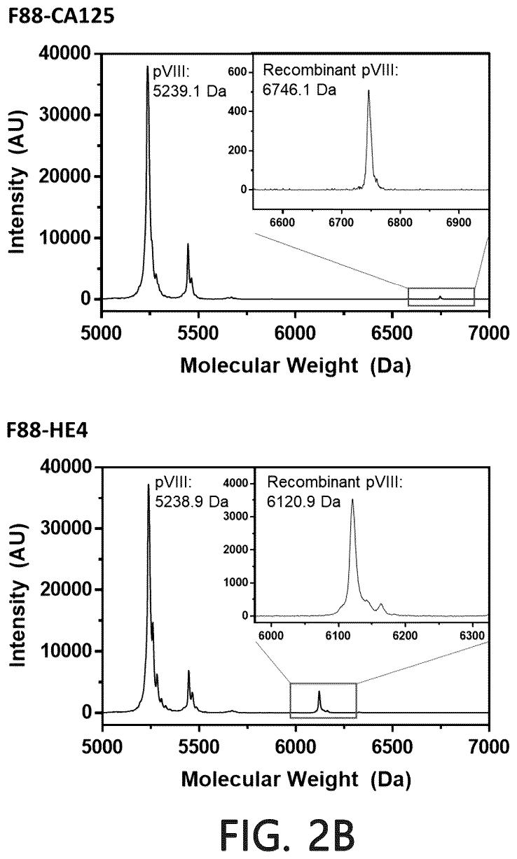

As a result, in SDS-PAGE, only the p8 protein band was confirmed in wild-type phage, but in F88-CA125 and F88-HE4 phages, it was confirmed that a thin p8 band modified with a biomarker-binding peptide appeared on top of the p8 protein band (FIG. 2A). Among these, in the case of F88-HE4 phage, the size of the introduced biomarker-binding peptide is small (total of 10 amino acids introduced), so it is difficult to clearly separate the band size, but by confirming the molecular weight through MALDI-TOF analysis, it was confirmed that the biomarker-binding peptide was well expressed on the phage surface by IPTG induction (FIG. 2B).

### Example 3. Mass Production of F88-CA125, F88-HE4 Phages and Fabrication of Self-Assembled Phage Nanostructures

To mass produce F88-CA125 and F88-HE4 phages, the phages were cultured in 800 mL LB medium inoculated with XL-1 Blue at 225 rpm at 37° C. for 9 hours. The culture medium was centrifuged at 12.1k g for 20 minutes at 4° C. to remove the bacterial pellet, and then, to obtain phages in the upper layer, ⅕ volume of PEG-NaCl solution was added to the supernatant and mixed well. When stored overnight in a refrigerator, the phage precipitated, so it was centrifuged at 15.9 k g for 20 minutes at 4° C. to obtain a phage pellet. Since the phage pellet still contained bacteria, the phage pellet was redispersed in TBS buffer and then centrifuged to remove only the bacterial pellet. This process was repeated 2 to 3 times to obtain cleanly purified phage.

A desired substrate, e.g., a gold-coated Si wafer, was cut into a desired size (typically 2×0.5 cm) using a diamond-tipped glass scribe, cleaned the wafer by blowing nitrogen gas, and then the gold-coated Si wafer was mounted on a syringe pump. Purified phages were suspended in TBS-T (12.5 mM Tris-HCl, pH 7.5, 37.5 mM NaCl, 0.05% Tween), loaded with substrate, and pulled using a pre-programmed syringe pump (KD Scientific) according to the manufacturer's instructions. Phage was coated on the wafer by gradually increasing the speed of the support on which the gold-coated wafer was fixed to 30, 40, 50, and 60 μm/min.

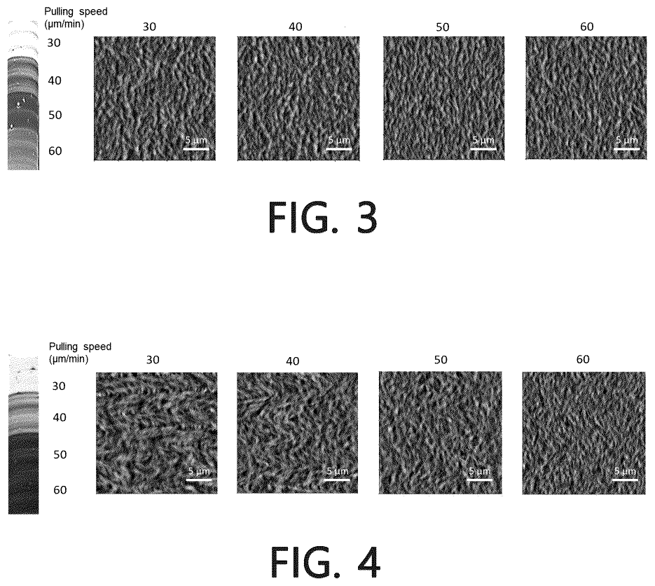

As a result, the sensing platform for CA125 detection made with F88-CA125 phage was confirmed to have different colors of the phage film depending on the pulling speed, and it was confirmed that as the pulling speed increased, the bundle size of the phage nanostructure gradually decreased (FIG. 3).

In addition, the sensing platform for HE4 detection made with F88-HE4 phage was also confirmed to have different colors of the phage film depending on the pulling speed, and as the pulling speed increased, the bundle size of the phage nanostructure gradually decreased (FIG. 4).

### Example 4. Color Change Analysis Using Smartphone Application

By analyzing color changes using a smartphone application, whether cancer diagnosis using CA125 and HE4 is possible was identified (see Oh, JW., Chung, WJ., Heo, K. et al. Biomimetic virus-based colourimetric sensors. Nat Commun 5, 3043 (2014)). With the above method, when taking a picture of the produced phage-coated film by linking it to a smartphone app that analyzes R.G.B. values, the color is automatically converted into R.G.B. values, allowing to compare the amount of color change.

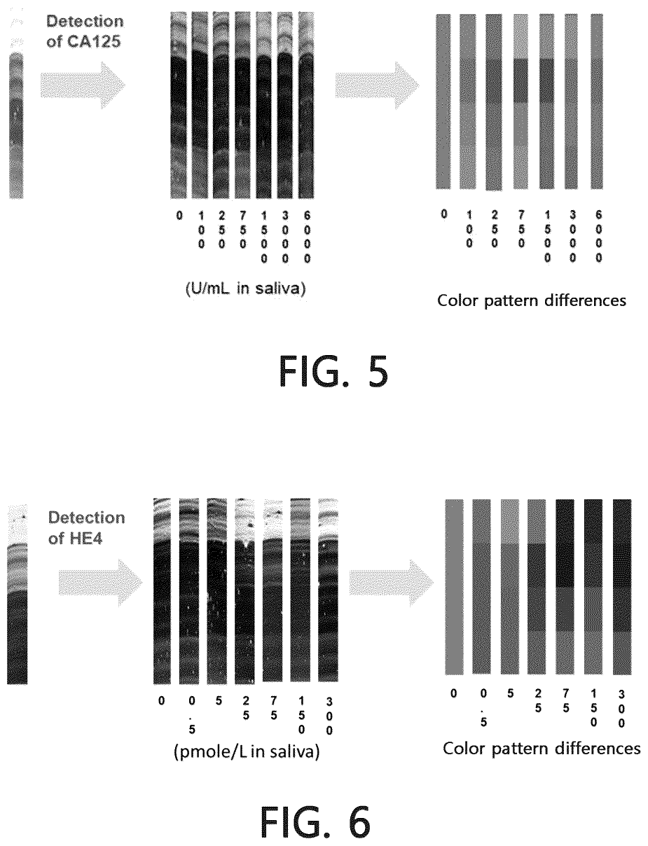

A detection experiment was conducted by spiking CA125 in normal saliva using the F88-CA125 phage nanostructure produced in Example 3. The color change was quantitatively expressed through RGB (Red, Green, Blue) pattern analysis, and to correct this, the color change was analyzed after conversion to Gray color. CA125 was spiked into normal saliva, and detection experiments were conducted for CA125 concentrations of 0, 100, 250, 750, 1500, 3000, and 6000 Unit/mL. It is known that if CA125 in saliva is more than or equal to 3000 Unit/mL, there is a high possibility of ovarian cancer (Oncology and radiotherapy, 2015), and according to the experimental results, it was confirmed that the nanostructure of the present invention was sufficiently detectable for cancer diagnosis (FIG. 5).

In addition, a detection experiment was conducted by spiking HE4 in normal saliva using the F88-HE4 phage nanostructure produced in Example 3. The color change was quantitatively expressed through RGB (Red, Green, Blue) pattern analysis, and to correct this, the color change was analyzed after conversion to Gray color. HE4 was spiked into normal saliva, and detection experiments were conducted for HE4 concentrations of 0, 0.5, 5, 25, 75, 150, and 300 pmole/L. It is known that HE4 in saliva is less than or equal to 150 pmole/L in normal patients (Oncology and radiotherapy, 2015), and in patients with ovarian cancer, HE4 is present in higher concentrations than this, so according to the experimental results, it was confirmed that the nanostructure of the present invention was sufficiently detectable for cancer diagnosis (FIG. 6).

### Example 5. CA125 and HE4 Multi-Detection Biosensors

F88-CA125 and F88-HE4 phages were mixed 1:1 to create a suspension, and bacteriophage nano self-assembly was produced in the same manner as Example 3 and Example 4.

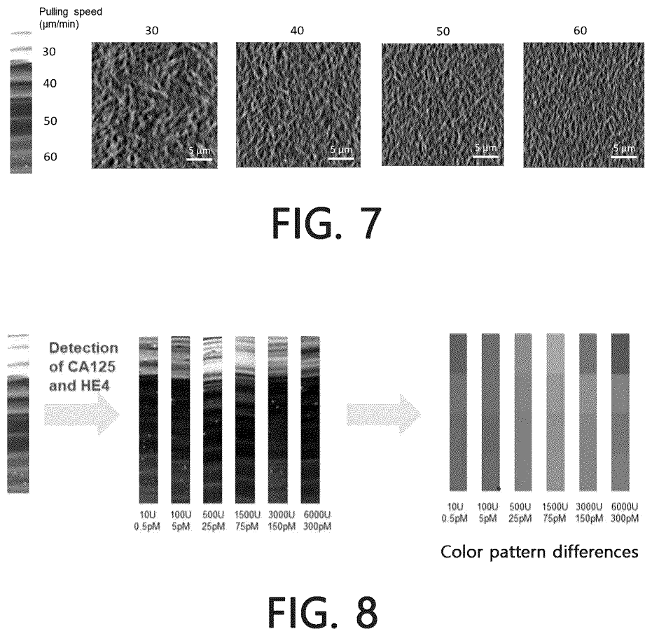

In nano self-assembly produced by mixing bacteriophages, it was confirmed that the color of the phage film appeared differently depending on the pulling speed, and it was confirmed that the bundle size of the phage gradually decreased (FIG. 7).

In addition, color changes could be expressed quantitatively through RGB (Red, Green, Blue) pattern analysis using a smartphone application, to correct this, the color change was analyzed after conversion to Gray color, and as a result, it was confirmed that CA125 and HE4 could be effectively simultaneously detected in a mixture of CA125 concentrations of 10 to 6000 U/mL and HE4 concentrations of 0.5 to 300 pM (FIG. 8).

As above, specific parts of the present invention have been described in detail, and it will be clear to those skilled in the art that these specific techniques are merely preferred embodiments and do not limit the scope of the present invention. Accordingly, the substantial scope of the present invention will be defined by the appended claims and their equivalents.

What is claimed is:

1. A peptide represented by SEQ ID NO: 2 that specifically binds to HE4.

2. The peptide of claim 1, wherein the peptide is a peptide for detecting ovarian cancer.

3. A composition for diagnosing ovarian cancer, comprising the peptide of claim 1.

4. A bacteriophage in which the peptide of claim 1 is expressed on its outer coat.

5. The bacteriophage of claim 4, wherein the bacteriophage is M13 or F88 phage.

6. A bacteriophage nano self-assembly produced by loading a substrate in the suspension of the bacteriophage of claim 4 and pulling the substrate vertically at a speed of 30 to 60 μm/min.

7. The bacteriophage nano self-assembly of claim 6, wherein the substrate is a gold-coated silicon substrate.

8. A method for diagnosing ovarian cancer, comprising:
(a) contacting a sample separated from a subject with the bacteriophage nano self-assembly of claim 6;
(b) analyzing a color change pattern of the bacteriophage nano self-assembly of step (a); and
(c) comparing the color change pattern of step (b) with the color change pattern of a control group to determine whether ovarian cancer has occurred.

9. The method of claim 8, wherein the sample is selected from the group consisting of whole blood, leukocytes, peripheral blood mononuclear cells, buffy coat, plasma, serum, sputum, tears, mucus, nasal washes, nasal aspirate, breath, urine, semen, saliva, peritoneal washings, ascites, cystic fluid, meningeal fluid, amniotic fluid, glandular fluid, pancreatic fluid, lymph fluid, pleural fluid, nipple aspirate, bronchial aspirate, synovial fluid, joint aspirate, organ secretions, cells, cell extract, and cerebrospinal fluid obtained from a separated organ, tissue, cell, or subject.

10. The method of claim 8, wherein the color change pattern in step (b) is an RGB (Red, Green, Blue) pattern.

11. The method of claim 10, wherein the RGB (Red, Green, Blue) pattern in step (b) is analyzed for the color change pattern after conversion to black and white.

12. A method for diagnosing ovarian cancer, comprising:
comparing the expression level of HE4 in a subject requiring ovarian cancer diagnosis with a control group using the peptide of claim 1.
